(19) United States
(12) Patent Application Publication
(10) Pub. No.: US 2024/0261138 A1
(43) Pub. Date: Aug. 8, 2024

(54) ROD INSERTER SYSTEM AND METHODS

(71) Applicant: Genentech, Inc., South San Francisco, CA (US)

(72) Inventors: Joshua David HORVATH, San Ramon, CA (US); Anna Lucia VOGEL, San Francisco, CA (US); Rachel Paige ARNOTT, San Francisco, CA (US); Ariel Ephraim WAITZ, Minnetonka, MN (US); Christopher Mark PAVLOFF, San Francisco, CA (US); William Churchill Taliaferro BURKE, Boulder, CO (US); Ndungu MUTURI, Oakland, CA (US); Christopher R. MCCASLIN, Portland, OR (US)

(21) Appl. No.: 18/407,243

(22) Filed: Jan. 8, 2024

Related U.S. Application Data
(63) Continuation of application No. PCT/US2022/036276, filed on Jul. 7, 2022.
(60) Provisional application No. 63/203,086, filed on Jul. 7, 2021.

(51) Int. Cl. A61F 9/00

(52) U.S. Cl. CPC A61F 9/0017; A61F 9/0026

(57) ABSTRACT

Provided herein are devices and methods for delivering medical implants into a patient, such as intravitreally. In one exemplary implementation, an implant delivery device includes an elongated body, cannula, mandrel, front shuttle, dispense button and leaf spring. The cannula is carried by the body and configured to receive an implant. The mandrel is slidably received within the cannula. The front shuttle is coupled to the mandrel. The leaf spring has at least a portion that spans between the dispense button and the front shuttle. The delivery device is configured to be operated by a user moving the dispense button from an extended position to a depressed position, thereby deflecting the leaf spring and urging the front shuttle towards the distal end of the body and causing the mandrel to move from a retracted position to a distally advanced position to eject the implant from a distal portion of the cannula.

### CROSS REFERENCE TO RELATED APPLICATIONS

This application is a continuation of International Application No. PCT/US2022/036276, filed Jul. 7, 2022 which claims the benefit of U.S. Provisional Application No. 63/203,086, filed Jul. 7, 2021, each of which is herein incorporated by reference in its entirety for all purposes.

### INCORPORATION BY REFERENCE

All publications and patent applications mentioned in this specification are incorporated herein by reference in their entirety to the same extent as if each individual publication or patent application was specifically and individually indicated to be incorporated by reference.

### BACKGROUND

The present disclosure relates to drug delivery systems and methods that can be used to introduce implants containing therapeutic or active agents, including bioerodible implants, into various locations within a patient, such as the eye, including into the vitreous of the eye.

A primary difficulty in treating diseases of the eye is the inability to introduce drugs or therapeutic agents into the eye and maintain these drugs or agents at a therapeutically effective concentration in the eye for the necessary duration. Systemic administration may not be an ideal solution because unacceptably high levels of systemic dosing are often needed to achieve effective intraocular concentrations, thus increasing the incidence of unacceptable side effects of the drugs. Simple ocular instillation or application is not an acceptable alternative in many cases because the drug may be quickly washed out by tear-action or may otherwise be depleted from the eye into the general circulation. Available methods make it difficult to maintain therapeutic levels of drug for adequate time periods.

Efforts to address this problem have led to the development of drug delivery devices, or implants, which can be implanted into the eye such that a controlled amount of desired drug can be released constantly over a period of several days, weeks, or even months. Many such devices have been previously reported. See, for example, U.S. Pat. No. 4,853,224, which discloses biocompatible implants for introduction into an anterior segment or a posterior segment of an eye for the treatment of an ocular condition. In addition, U.S. Pat. No. 5,164,188 discloses a method of treating an ocular condition by introduction of a biodegradable implant comprising drugs of interest into the suprachoroidal space or pars plana of the eye. See also U.S. Pat. Nos. 5,824,072; 5,476,511; 4,997,652; 4,959,217; 4,668,506; and 4,144,317. Other methods include anchoring a plug or tack containing a drug into the sclera of the eye (see, e.g., U.S. Pat. No. 5,466,233).

Various sites exist in the eye for implantation of a drug delivery device or implant, such as the vitreous of the eye, anterior or posterior chambers of the eye, or other areas of the eye including intraretinal, subretinal, intrachoroidal, suprachoroidal, intrascleral, episcleral, subconjunctival, intracorneal or epicorneal spaces. Wherever the desired location of implantation, typical methods of implantation all require relatively invasive surgical procedures, pose a risk of excessive trauma to the eye, and require excessive handling of the implant. For example, in a typical method for placement in the vitreous, an incision is made through the sclera, and the implant is inserted into and deposited at the desired location in the vitreous, using forceps or other like manual grasping device. Once deposited, the forceps (or grasping device) are removed, and the incision is sutured closed. Alternatively, an incision can be made through the sclera, a trocar can be advanced through the incision and then the implant can be delivered through the trocar. Similar methods can be employed to deliver implants to other locations, e.g., implantation in the anterior chamber of the eye through an incision in the cornea.

There are numerous drawbacks of such techniques for implant delivery. Extensive handling of the implant is necessitated in these techniques, creating a risk that the implant will be damaged in the process. Many implants are polymer-based and are relatively fragile. If portions of the implants are damaged and broken-off, the effective therapeutic dose delivered by the implant once placed will be significantly altered. In addition, it becomes inherently difficult using these methods to achieve reproducible placement from patient to patient. Additionally, all of these techniques require an incision or puncture in the eye large enough to require suturing. Thus, such techniques are typically performed in a surgical setting.

Many considerations affect the design and efficacy of an implant delivery device. First, it is important to ensure that the implant is consistently delivered to the subject with each application. Second, because implant therapy often requires numerous applications, the cost of providing the implant should also be considered.

Based on the foregoing, a need for a more facile, convenient, less invasive, and less traumatic means for delivering implants into the eye and other anatomy remains. In addition, a need for a more controlled means of delivering implants also remains. The innovations described herein solve these unmet needs and provide additional advantages.

### SUMMARY OF THE DISCLOSURE

According to aspects of the present disclosure, an implant delivery device may be provided with an elongated body, a cannula, a mandrel, a front shuttle, a dispense button and a leaf spring. In some embodiments, the elongated body has a proximal end and a distal end. The cannula is carried by the body and has a distal portion extending from the distal end of the body. The cannula is sized and configured to receive an implant therein. The mandrel is slidably received within the cannula and movable from a retracted position, in which an implant is retained inside the cannula, to a distally advanced position, in which the implant is ejected from the cannula by the mandrel. The front shuttle is coupled to the mandrel and is configured to drive the mandrel from the retracted position to the distally advanced position. The dispense button is movable from an extended position, in which the mandrel remains in the retracted position, to a depressed position, in which the mandrel is moved toward the distally advanced position. At least a portion of the leaf spring spans between the dispense button and the front shuttle. The delivery device is configured to be operated by a user moving the dispense button from the extended position to the depressed position, thereby deflecting the leaf spring and urging the front shuttle towards the distal end of the body and causing the mandrel to move from the retracted position to the distally advanced position to eject the implant from the distal portion of the cannula.

In some embodiments, the dispense button is located on a top side surface of the elongated body and generally moves radially inward when moving from the extended position to the depressed position. The device may further comprise a rear shuttle that is separately movable with respect to the front shuttle. In some embodiments, the leaf spring spans between the front shuttle and the rear shuttle and has a mid-portion that contacts the dispense button. The leaf spring may be pivotably connected to the front shuttle with a first pin and pivotably connected to the rear shuttle with a second pin.

In some embodiments, the dispense button comprises a cam surface that contacts a mating cam surface on the rear shuttle to drive the rear shuttle distally when the dispense button is moving from the extended position to the depressed position, thereby urging the leaf spring, the front shuttle and the mandrel further towards the distal end of the body. The rear shuttle may comprise a locking feature that engages with a mating locking feature on the dispense button to lock the dispense button in the depressed position. In some embodiments, the front shuttle and the rear shuttle have portions that overlap one another. In these embodiments, the device further comprises a lock pin that extends from outside the elongated body and into the overlapping shuttle portions to prevent the front shuttle and the rear shuttle from moving relative to the body until the lock pin is removed.

In some embodiments, the device further comprises a cap configured to releasably cover the distal end of the body and the distal portion of the cannula when the cap is attached to the body. In these embodiments, the cap is configured to be locked onto the body until the lock pin is removed.

In some embodiments, the device further comprises a sharps protection slide that is movable from a retracted position to a distally extended position in which the slide at least partially covers a distal tip of the cannula. The sharps protection slide may cover a bottom portion of the cannula along a longitudinal length extending at least to the distal tip of the cannula and may leave a top portion visually exposed but not reachable by a flat object when the slide is in the extended position. In some embodiments, the slide is configured to be locked in the retracted position until the lock pin is removed.

In some embodiments, the shuttle and the leaf spring are configured to return the dispense button to the extended position if the dispense button is not fully moved to the depressed position. The device may be configured to emit a soft click when the dispense button reaches the depressed position.

In some embodiments, the cannula is provided with a retaining feature configured to releasably retain an implant within the cannula until the implant is pushed out by the mandrel. The retaining feature may comprise a cutout section in the cannula which exposes at least a portion of the implant when loaded in the cannula, and a retention member configured to extend into the cutout section and urge the implant against an opposite wall of the cannula. In some embodiments, the retention member is a flexible arm that puts a resilient force on the implant. The flexible arm may be mounted on a retention hub that concentrically surrounds a portion of the cannula. In some embodiments, the cutout section in the cannula has a longitudinal length that is nominally the same as a longitudinal length of the implant. The flexible arm may comprise an implant contact surface having a longitudinal length that is nominally the same as the longitudinal lengths of the implant and the cutout section. In some embodiments, the device further comprises a preloaded implant.

According to aspects of the present disclosure, an implant delivery device may be provided with an elongated body, a cannula, a mandrel, a front shuttle, a rear shuttle, a dispense button, a leaf spring, a cap, a sharps protection slide and a lock pin. In these embodiments, the elongated body has a proximal end, a distal end and a longitudinal axis. The cannula is carried by the body and has a distal portion extending from the distal end of the body. The cannula is sized and configured to receive an implant therein. A mandrel is slidably received within the cannula and movable from a retracted position, in which an implant is retained inside the cannula, to a distally advanced position, in which the implant is ejected from the cannula by the mandrel. The front shuttle is movable in a longitudinal direction and coupled to the mandrel. The front shuttle is configured to drive the mandrel distally from the retracted position to the distally advanced position. The rear shuttle is movable in a longitudinal direction separately from the front shuttle. The dispense button is located on a top side surface of the elongated body and is generally movable in a radially inward direction when moving from an extended position to a depressed position. The leaf spring has a first end pivotably connected to the front shuttle, a second end pivotably connected to the rear shuttle, and a convex mid-portion contacting a surface of the dispense button such that when the button is moved toward the depressed position, the leaf spring becomes less curved and urges the front shuttle apart from the rear shuttle. The dispense button comprises a cam surface that contacts a mating cam surface on the rear shuttle to drive the rear shuttle and leaf spring distally when the dispense button is moving towards the depressed position. The rear shuttle comprises a locking feature that engages with a mating locking feature on the dispense button to lock the dispense button in the depressed position. The cap is configured to releasably cover the distal end of the body and the distal portion of the cannula when the cap is attached to the body. The sharps protection slide is movable from a retracted position to a distally extended position in which the slide at least partially covers a distal tip of the cannula. The lock pin is configured to pass through the cap, the sharps protection slide, the rear shuttle and the front shuttle to prevent these components from moving until the lock pin is removed. The delivery device is configured to be operated by a user removing the lock pin, removing the cap, moving the dispense button from the extended position to the depressed position, thereby moving the rear shuttle distally and deflecting the leaf spring, both of which urge the front shuttle towards the distal end of the body and cause the mandrel to move from the retracted position to the distally advanced position to eject the implant from the distal portion of the cannula. The user may then move the sharps protection slide from the retracted position to the distally extended position.

In some embodiments, the first shuttle, the second shuttle and the leaf spring are configured to return the dispense button to the extended position if the dispense button is not fully moved to the depressed position. The device may be configured to emit a soft click when the dispense button reaches the depressed position.

In some embodiments, the device further comprises a retention hub that concentrically surrounds a portion of the cannula. In these embodiments, the retention hub has a flexible arm that extends through a cutout section in the cannula to resiliently urge an implant inside the cannula against an opposite wall of the cannula, thereby releasably retaining the implant within the cannula until the implant is pushed out by the mandrel.

According to aspects of the present disclosure, a method of delivering an implant includes the step of providing an implant delivery device. In this method, the implant delivery device includes an elongated body, a cannula, a mandrel, a front shuttle, a dispense button, a leaf spring, a cap and a lock pin. The elongated body has a proximal end and a distal end. The cannula is carried by the body, has a distal portion extending from the distal end of the body and carries an implant therein. The mandrel is slidably received within the cannula. The front shuttle is coupled to the mandrel. The dispense button is located on the elongated body. At least a portion of the leaf spring spans between the dispense button and the front shuttle. The cap is removably attached to the distal end of the body and covers the distal portion of the cannula when the cap is attached to the body. The lock pin is configured to pass through the cap and the front shuttle to prevent these components from moving until the lock pin is removed. The method further includes the steps of removing the lock pin, removing the cap and inserting a distal tip of the cannula into a patient. According to the method, the dispense button is moved from an extended position to a depressed position, thereby deflecting the leaf spring which urges the front shuttle towards the distal end of the body and causes the mandrel to move from a retracted position to a distally advanced position to eject the implant from the distal portion of the cannula.

In some embodiments, the method further comprises moving a sharps protection slide from a retracted position to a distally extended position in which the slide at least partially covers the distal tip of the cannula.

In some embodiments, the implant is ejected intravitreally.

### BRIEF DESCRIPTION OF THE DRAWINGS

The novel features of the disclosure are set forth with particularity in the claims that follow. A better understanding of the features and advantages of the present disclosure will be obtained by reference to the following detailed description that sets forth illustrative embodiments, in which the principles of the disclosure are utilized, and the accompanying drawings of which:

FIG. 1 is a perspective view of an exemplary embodiment of an implant delivery device constructed and operated according to aspects of the present disclosure;

FIG. 2 is a perspective view of the device shown in FIG. 1 with its interlock pin and cap removed;

FIG. 3 is a perspective view of the device shown in FIG. 1 with various components removed for clarity;

FIG. 4 is a side cross-sectional view of the device shown in FIG. 1;

FIG. 5 is an exploded view of the device shown in FIG. 1;

FIG. 6 is an enlarged perspective view showing various components located near the distal end of the device shown in FIG. 1;

FIG. 7 is a front cross-sectional view of the device shown in FIG. 1;

FIGS. 8-10 are a series of side cross-sectional views of the device shown in FIG. 1 as the implant dispense button is being depressed;

FIG. 11 is a perspective view of implant retaining components of the device shown in FIG. 1;

FIG. 12 is a side cross-sectional view of the implant retaining components shown in FIG. 11;

FIGS. 13-14 are side cross-sectional views of an alternative embodiment of implant retaining components;

FIG. 15 is an enlarged perspective view of the distal end of the device shown in FIG. 1 showing a cannula protection and sharps protection slide in a retracted position;

FIG. 16 is an enlarged perspective view of the distal end of the device shown in FIG. 1 showing a cannula protection and sharps protection slide in an extended position;

FIG. 17 is a side cross-sectional view of the device shown in FIG. 1 showing a cannula protection and sharps protection slide in a retracted position;

FIG. 18 is a side cross-sectional view of the device shown in FIG. 1 showing a cannula protection and sharps protection slide in an extended position;

FIG. 19 is a perspective view of a second exemplary embodiment of an implant delivery device constructed and operated according to aspects of the present disclosure;

FIG. 20 is a side cross-sectional view of the device shown in FIG. 19;

FIG. 21 is a perspective view showing inner components of the device shown in FIG. 19;

FIG. 22 is an exploded perspective view showing components of the device shown in FIG. 19; and

FIG. 23 is a side view showing an exemplary lock plate of the device shown in FIG. 19.

### DETAILED DESCRIPTION

Described herein are exemplary devices and methods for delivering rod-shaped implants subcutaneously, intravitreally, intramuscularly, intraarticularly, intravascularly or into other anatomy. In some embodiments, an implant delivery device includes an elongated body, cannula, mandrel, front shuttle, dispense button and leaf spring. The cannula is carried by the body and configured to receive an implant. The mandrel is slidably received within the cannula. The front shuttle is coupled to the mandrel. The leaf spring has at least a portion that spans between the dispense button and the front shuttle. The delivery device is configured to be operated by a user moving the dispense button from an extended position to a depressed position, thereby deflecting the leaf spring and urging the front shuttle towards the distal end of the body and causing the mandrel to move from a retracted position to a distally advanced position to eject the implant from a distal portion of the cannula. In some embodiments, multiple implants may be dispensed from a single device, one at a time.

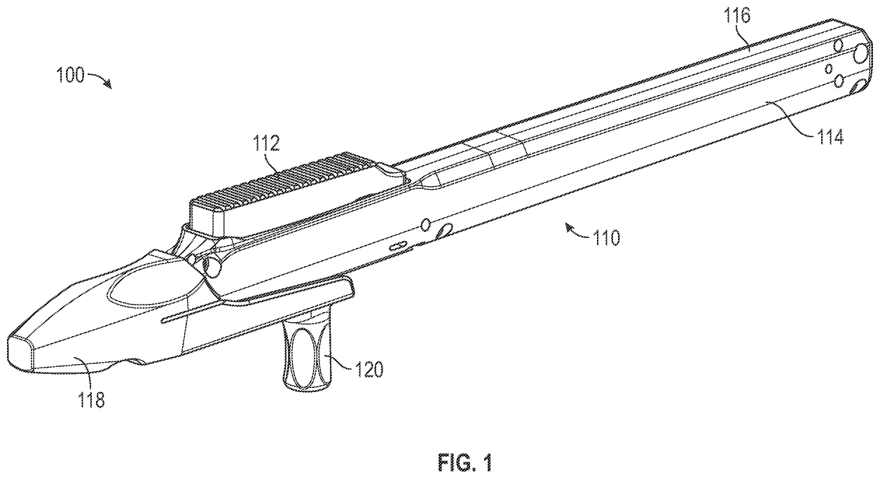

Referring to FIG. 1, an exemplary embodiment of an implant delivery device 100 is provided. Device 100 includes an elongated enclosure 110 having an implant dispense button 112 extending from the top of its distal end. Enclosure 110 may be formed from a left clamshell 114 and a right clamshell 116, as shown. A cap 118 may be provided to cover the distal end of enclosure 110 when not in use. In this exemplary embodiment, an interlock pin and retainer 120 is provided to lock cap 118 in place, as well as provide additional locking features which will be subsequently described.

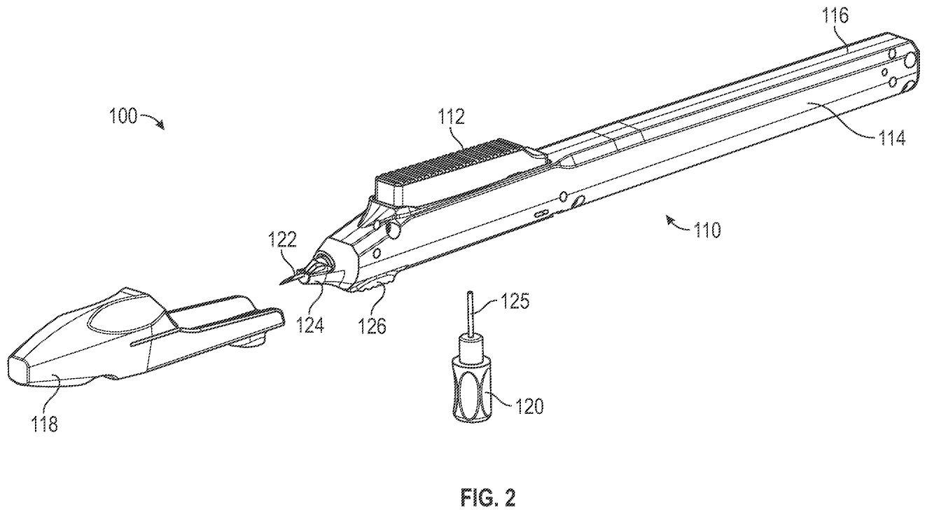

Referring to FIG. 2, implant delivery device 100 is shown with interlock pin retainer 120 and cap 118 removed, thereby exposing cannula 122 and cannula protection and sharps protection slide 124. Protection slide 124 includes a thumb or finger grip 126 for actuation, as will be subsequently described in more detail. Interlock pin 125 may be seen in FIG. 2 projecting upwardly from pin retainer 120

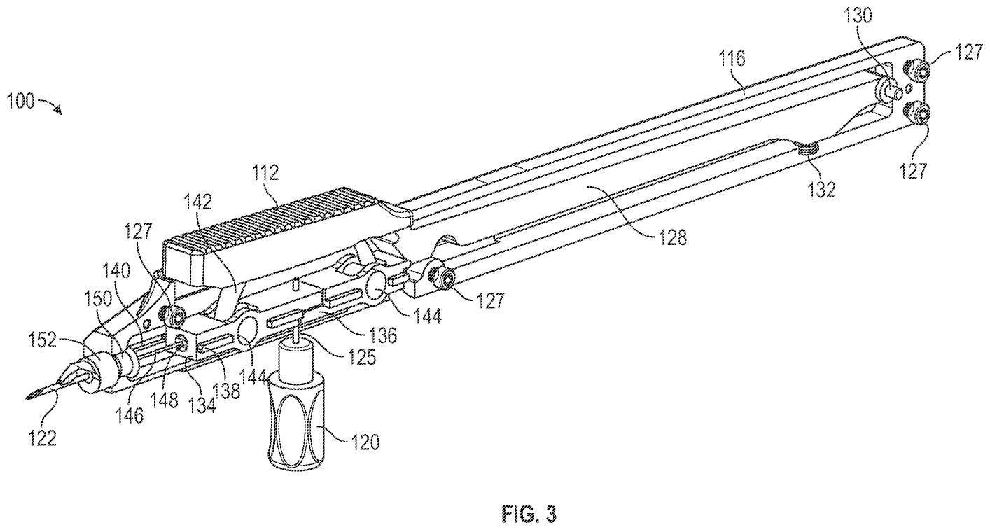

Referring to FIG. 3, implant delivery device 100 is shown with its cap, cannula protection slide and left clamshell removed so that its internal components may be seen. When device 100 is assembled, four fasteners 127 may be used to secure the two halves of the enclosure together.

In this exemplary embodiment, implant dispense button 112 is formed on the distal end of button lever 128 which resides inside the enclosure of device 100. The proximal end of lever 128 is pivotably mounted on pin 130 which extends between right clamshell 116 and the opposite clamshell (not shown.) This arrangement allows dispense button 112 to move from an extended position as shown in FIG. 3, to a depressed position as shown in FIG. 10. A compression spring 132 may be located between lever 128 and the enclosure to bias button 112 towards the extended position.

A front shuttle 134 and a rear shuttle 136 may be provided within the enclosure of device 100 as shown. Each shuttle may be provided with laterally extending ribs 138 on each side for engaging in longitudinally extending slots 140 located on each half of the enclosure. This arrangement allows front shuttle 134 and a rear shuttle 136 to independently move longitudinally within the enclosure. The rear section of front shuttle 134 and the front section of rear shuttle 136 may be provided with overlapping portions, as shown in FIG. 3, like a lap joint. This allows interlock pin 125 to pass through the device enclosure and both front shuttle 134 and rear shuttle 136, thereby locking the shuttles in place relative to the enclosure until pin 125 is removed.

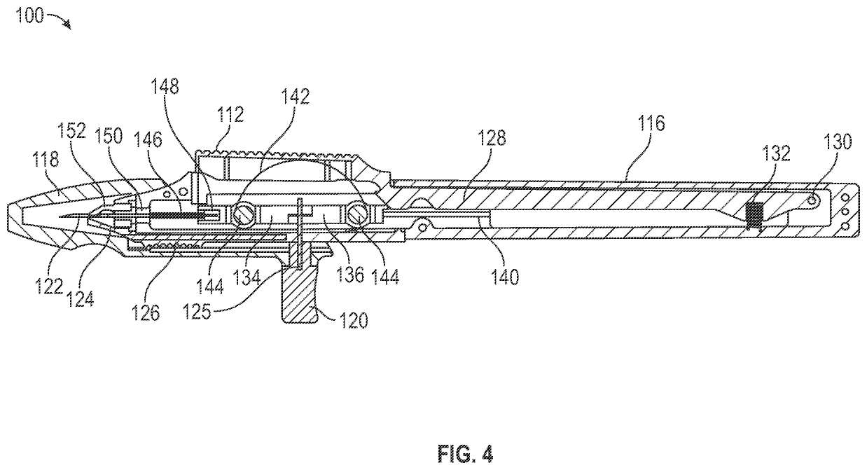

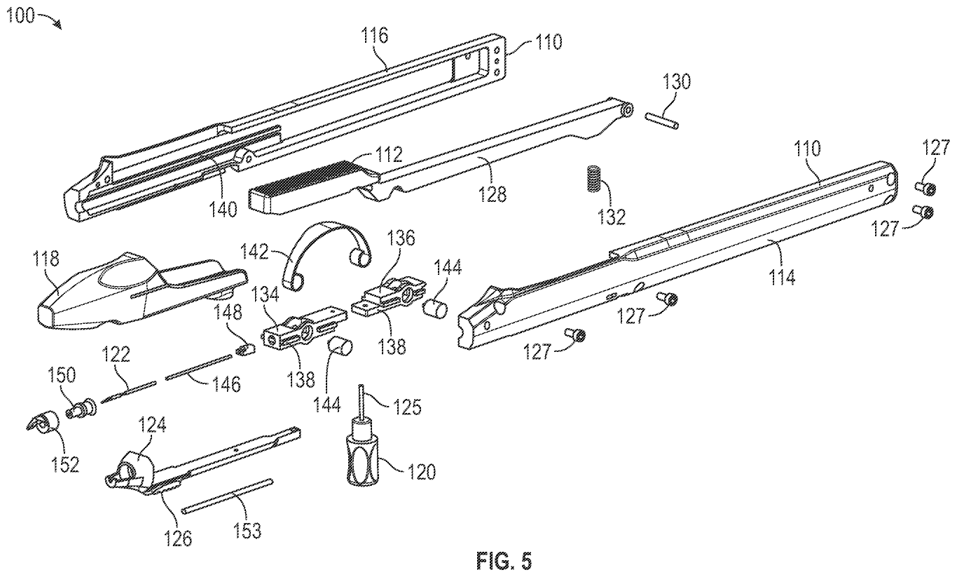

A leaf spring 142 may be provided that spans between front shuttle 134 and rear shuttle 136. Each end of leaf spring 142 may be pivotably connected to one of the shuttles with a shuttle pin 144 (see also FIG. 5.) A center portion of leaf spring 142 may be received in a recess on the bottom of button 112, such that a bottom surface of button 112 contacts a top surface of leaf spring 142 (see also FIG. 4.) In this embodiment, as best seen in FIG. 5, leaf spring 142 is wider at its mid-section than it is at its end sections.

Mandrel 146 may be rigidly connected to front shuttle 134 with a mandrel bushing 148 (see also FIG. 5.) Mandrel 146 is slidably received in cannula 122 which is rigidly affixed to the distal end of the enclosure of device 100 by cannula mount 150. Mandrel 146 serves to push an implant (not shown in FIG. 3) out of cannula 122 when mandrel 146 is driven distally by front shuttle 134. Implant retention hub 152 serves to releasably retain the implant in cannula 122, as will be subsequently described in more detail.

Referring to FIG. 4, a side cross-sectional view is provided to show further details of the previously described features of device 100.

Referring to FIG. 5, an exploded view is provided to show further details of the previously described features of device 100. FIG. 5 further shows reinforcement pin 153 which is embedded within protection slide 124 to provide it with additional rigidity.

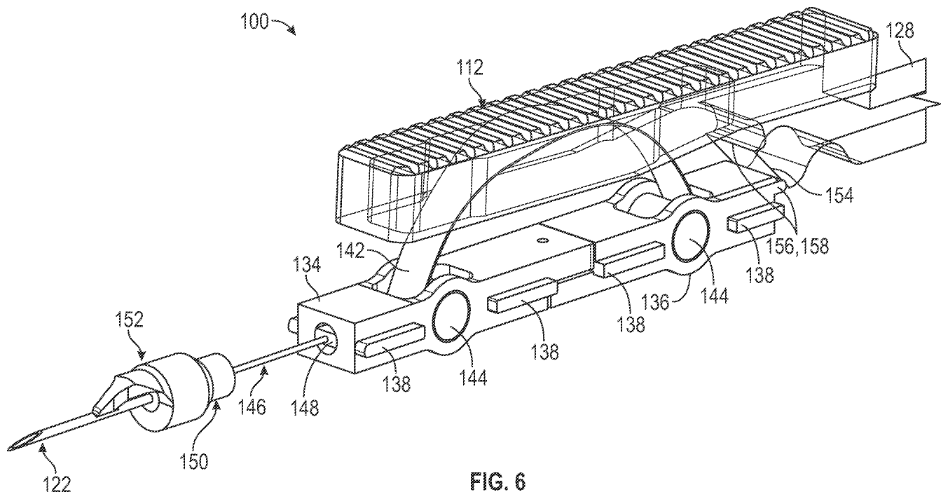

Referring to FIG. 6, an enlarged, partially transparent perspective view is provided to show further details of the components located near the distal end of device 100. FIG. 6 further shows ramped surface 154 located on dispense button lever 128, which contacts the proximal end of rear shuttle 136 for driving it in the forward/distal direction when dispense button 112 is depressed. A detent notch 156 may be located at the top of ramped surface 154 for engaging with a mating feature 158 on the proximal end of rear shuttle 136 when button 112 reaches the end of its downward stroke.

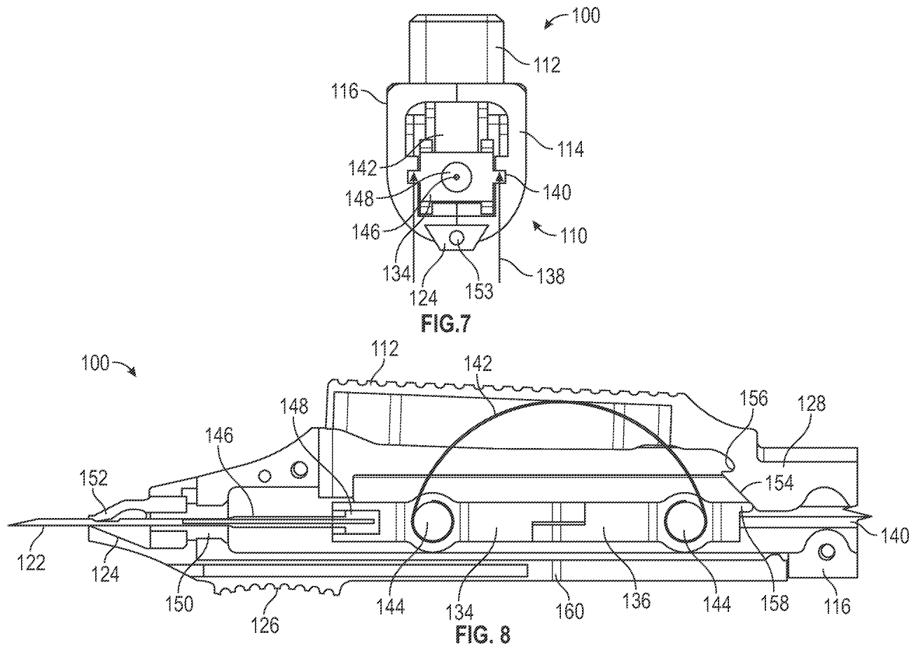

Referring to FIG. 7, a distal end cross-sectional view of device 100 is provided to show further details of the components located near the distal end of device 100.

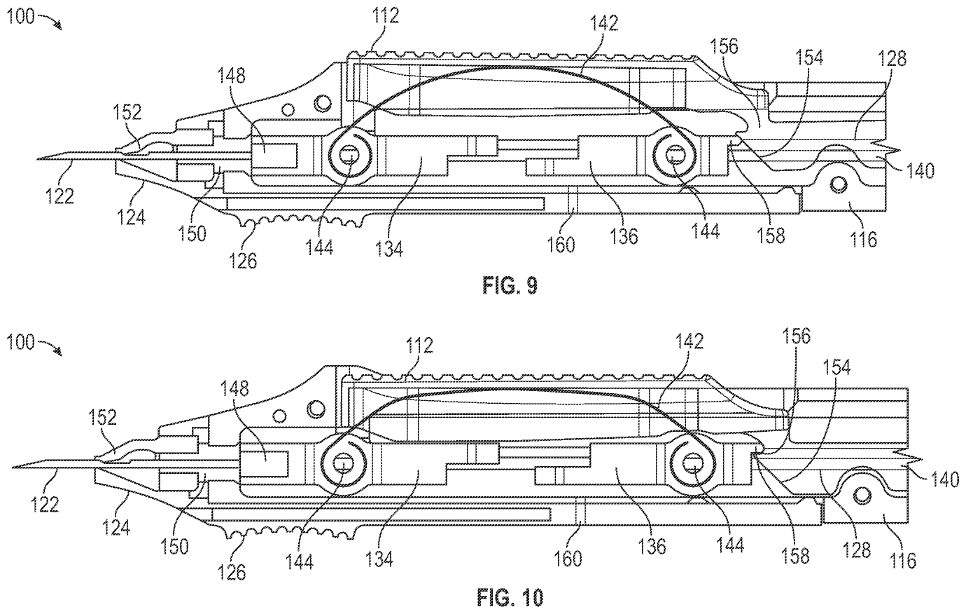

Referring to FIGS. 8-10, the implant dispensing operation of device 100 will now be described. FIG. 8 shows button 112 in a vertically extended position before being depressed, FIG. 9 shows button 112 traveling towards a depressed position, and FIG. 10 shows button 112 in a fully depressed, end of stroke locked position (after the implant has been fully ejected from cannula 122.) Referring first to FIG. 8, front shuttle 134 and rear shuttle 136 are shown adjacent to one another in a proximal-most position. In this position, leaf spring 142 is in a related state. Once interlock pin 125 (shown in FIG. 2) has been removed from hole 160, front shuttle 134 and rear shuttle 136 are free to move longitudinally within device enclosure 116. Front shuttle 134 and rear shuttle 136 are prevented from moving proximally (to the right in FIGS. 8-10) by the rear edge 158 of rear shuttle 136 abutting against ramped surface 154. When button 112 is pushed downwardly (into the device enclosure), ramped surface 154 slides along the rear edge 158 of rear shuttle 136, thereby urging rear shuttle 136 forward (distally, to the left.) At the same time, the bottom surface of button 112 pushes down on leaf spring 142, causing it to flatten out. The combination of rear shuttle 136 being urged forward and leaf spring 142 flattening out causes front shuttle 134 to move forward, as shown in FIG. 9. In this exemplary embodiment, front shuttle 134 moves forward at a faster rate and greater distance than rear shuttle 136, as can be appreciated by comparing the interlock pin hole locations of the shuttles compared with their starting locations over hole 160 in enclosure 116. In some embodiments, front shuttle 134 moves distally at least four times as far rear shuttle 136. In other embodiments, front shuttle 134 moves distally at least two times, at least three times, at least five times or at least six times as far rear shuttle 136. As front shuttle 134 moves distally, it drives mandrel 146 distally further into cannula 122. This in turn drives an implant located in cannula 122 out of the distal end and into the target anatomy of the patient. This arrangement allows the downward/inward motion of button 112 to be magnified into a larger stroke of mandrel 146.

As can be seen in FIG. 9, when button 112 is nearing the end of its stroke, the top of ramped surface 154 approaches mating feature 158 on rear shuttle 136. When button 112 is pushed further downward, mating feature 158 drops into detent notch 156 on button arm 128 as shown in FIG. 10, thereby locking button 112 in a depressed position. This signals to the device user that the button has reached the end of its stroke and confirms that the implant has been ejected from cannula 122. Detent notch 156 and mating feature 158 may be configured to emit a soft click, providing an audible and or tactile confirmation of implant ejection, in addition to the visual confirmation.

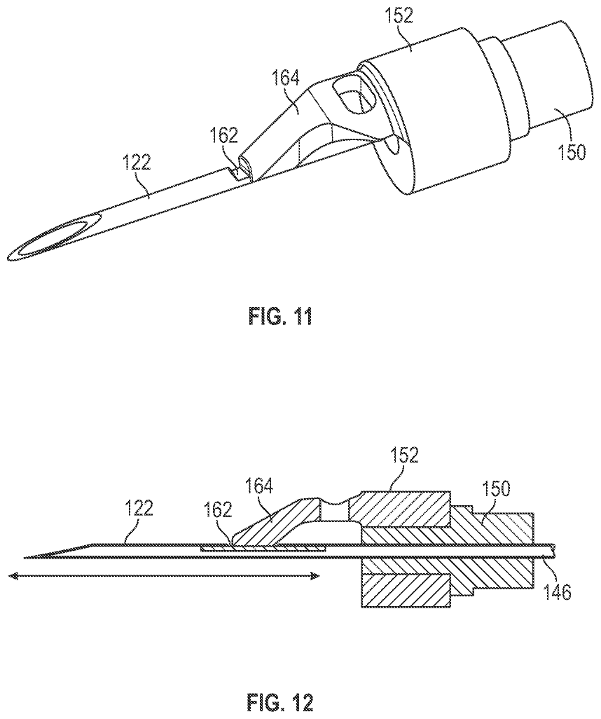

Referring to FIGS. 11 and 12, implant storage position and retention features are shown and described. In some embodiments, device 100 is a single-use disposable device that is provided to medical practitioners with an implant 162 preloaded within cannula 122. Cannula mount 150 may be provided to mount cannula 122 to the distal end of the device enclosure, as previously described. To inhibit implant 162 from falling out of cannula 122 due to device tilt, implant retention hub 152 may be provided over cannula mount 150 as shown. Implant retention hub 152 may be provided with a distally extending arm 164 which serves to releasably retain implant 162 in cannula 122. In this exemplary embodiment, the distal end of arm 164 passes through a window formed in cannula 122 to contact implant 162. Arm 164 may include a flexure portion such that arm 164 is able to maintain a light downward/inward force on implant 162 to hold the implant in place. When mandrel 146 pushes implant 162 distally, arm 164 may flex slightly upward to allow the implant and mandrel to move past it. In this exemplary embodiment, mandrel 146 has an 11 mm stroke length.

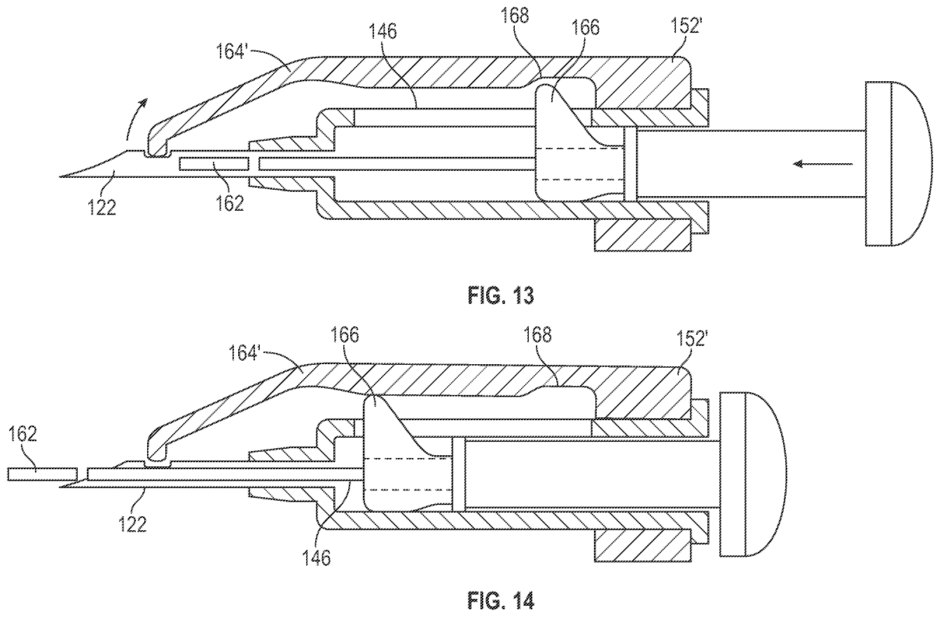

Referring to FIGS. 13 and 14, an alternative embodiment for an implant retention hub 152′ is provided. Implant retention hub 152′ may be constructed and operate in a similar manner to implant retention hub 152 described above in relation to FIGS. 11 and 12. A front shuttle or other component used to drive mandrel 146 distally may be provided with an upwardly extending cam 166. When cam 166 is in a proximal position as shown in FIG. 13, it resides in a recess 168 located on the bottom surface of retention arm 164′ such that it does not apply a force to arm 164′. When the front shuttle moves distally to eject implant 162, cam 166 contacts the bottom side of arm 164′ and urges it upward such that the distal tip of arm 164′ moves away from implant 162. This arrangement may provide additional advantages, such as lower frictional forces when dispensing implant 162 through cannula 122.

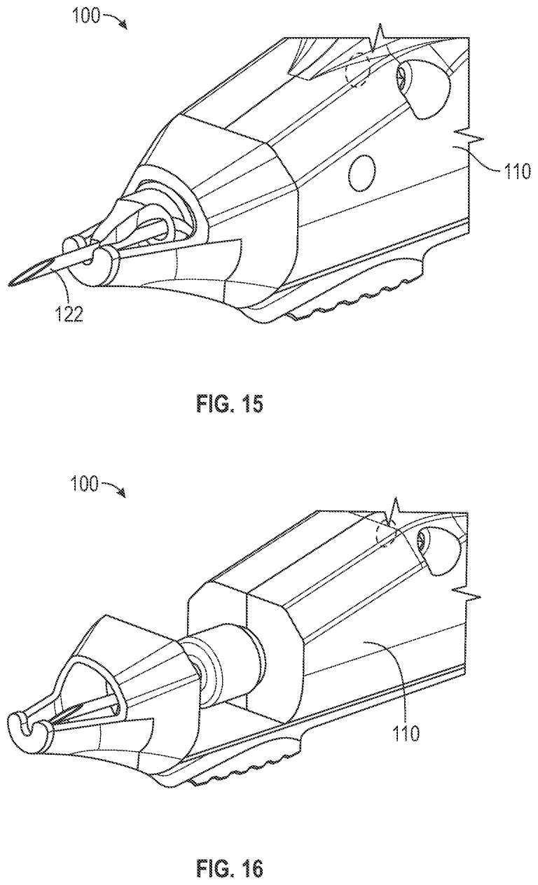

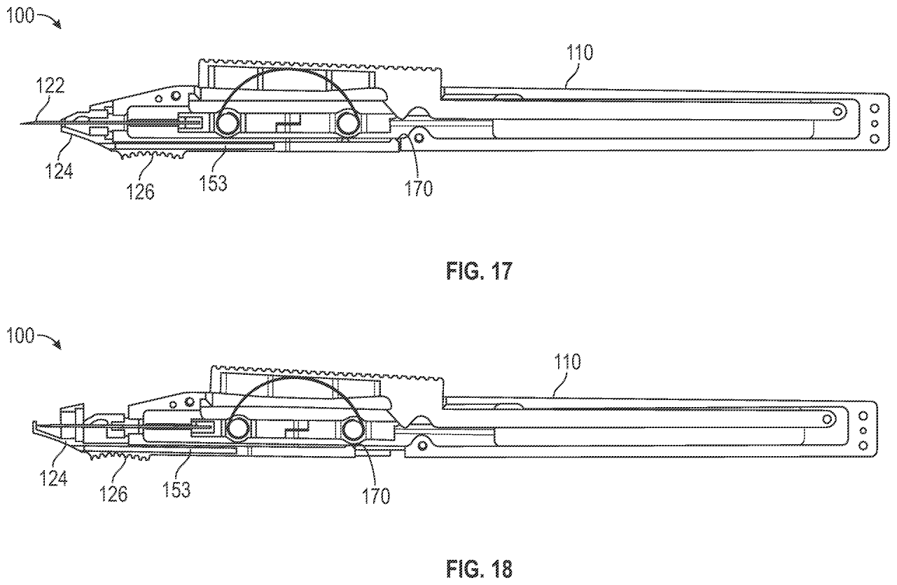

Referring to FIGS. 15-18, additional details of cannula protection and sharps protection slide 124 are provided. The proximally extending (right) portion of slide 124 may be provided with a dovetail configuration (best seen in FIG. 7) so that it may be slidably retained on the bottom of the device enclosure 110. Slide 124 may be moved between a retracted position as shown in FIGS. 15 and 17, and an extended position as shown in FIGS. 16 and 18. A detent 170 may be provided on slide 124 to hold it in the retracted and extended positions. As previously mentioned, a thumb grip 126 may be provided for moving slide 124, and a stiffening rod 153 may be embedded in slide 124 to provide additional rigidity, particularly when slide 124 is extended. In this exemplary embodiment, cannula 122 extends 6.3 mm beyond the distal surface of slide 124 when the slide is in the retracted position. When slide 124 is in the extended position, cannula 122 may be seen from above device 100 but protection slide 124 prevents cannula 122 from contacting people or objects. A silicone over-molded surface may be provided on the distal end of slide 124 such that it may serve as a depth gage or stop when inserting cannula 122 into a patient.

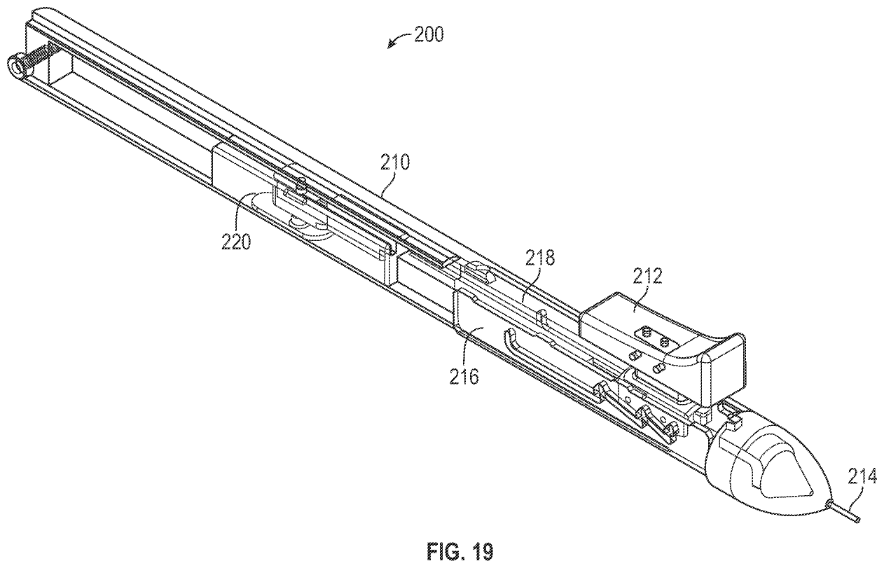

Referring to FIGS. 19-23, a second exemplary embodiment of an implant delivery device 200 is provided. Device 200 is configured to deliver multiple pre-loaded implants, one at a time. In the configuration shown, device 200 can deliver two implants. The concepts taught by this embodiment can be expanded to create devices that deliver three, four or more implants.

Referring first to FIG. 19, implant delivery device 200 may be provided with an enclosure 210 (only the left side of the enclosure is shown for clarity.) An implant dispense button 212 may be provided on top of enclosure 210. Button 212 is configured to be downwardly depressed and slid longitudinally. As with the previous embodiment, a cannula 214 with a preloaded implant extends from the distal end of enclosure 210. A pair of lock plates 216 and 218 may be rigidly mounted within enclosure 210 in a spaced apart configuration. A cantilever shuttle 220 is configured for longitudinal movement within enclosure 210.

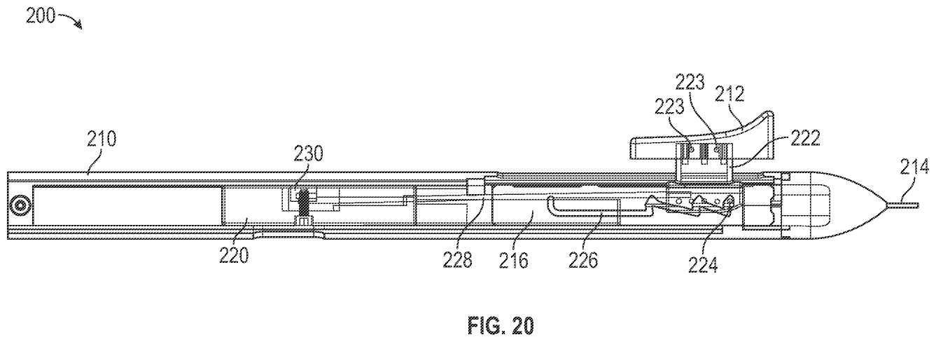

Referring next to FIG. 20, a switch shuttle 222 is rigidly pinned to dispense button 212 with pins 223 and extends downwardly therefrom into enclosure 210 between lock plates 216 and 218 (lock plate 218 not shown in FIG. 20.) A pair of pins 224 protrude laterally from each side of switch shuttle 222 and engage a slot 226 in each lock plate 216. A pair of cantilever rod springs 228 extends distally from shuttle 220 and biases button 212 upwardly/outwardly. Cantilever rod springs 228 may be rigidly attached to shuttle 220 with cantilever trap 230, and their opposite ends are rigidly attached to switch shuttle 222. With this arrangement, button 212 is able to be depressed against the spring bias of cantilever rod springs 228, and can move cantilever shuttle 220 longitudinally through switch shuttle 222 and cantilever rod springs 228.

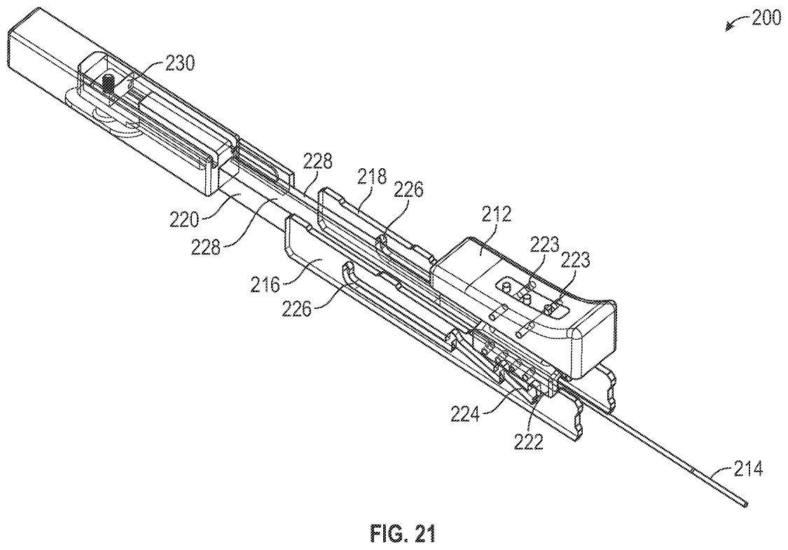

Referring to FIG. 21, a perspective view of device 200 is provided with the device enclosure removed for clarity and showing interior components of device 200.

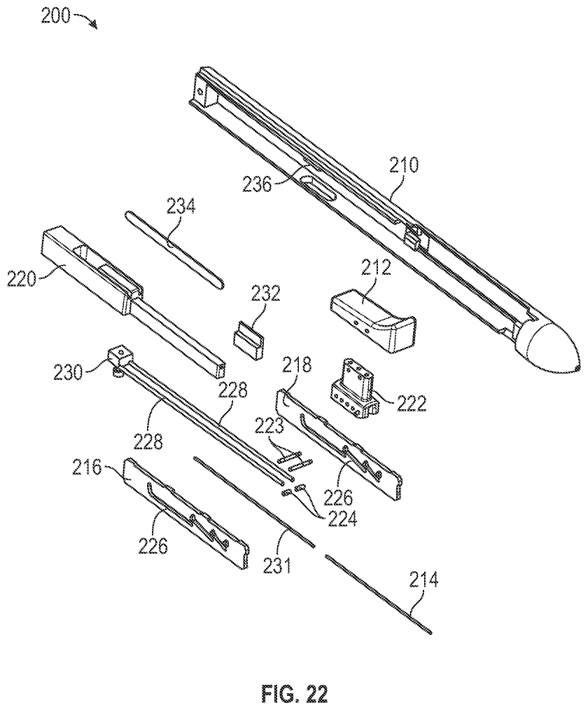

Referring to FIG. 22, an exploded view of device 200 is provided. FIG. 22 further shows mandrel 231 that rigidly attaches to the distal end of shuttle 220 and is received within cannula 214 for pushing out the implants one at a time. Extension block 232 attaches to shuttle 220 and supports part of sticker 234. Sticker 234 has various colors and or symbols that show through window 236 in enclosure 210 in sequence to show the status of device 200 to a user.

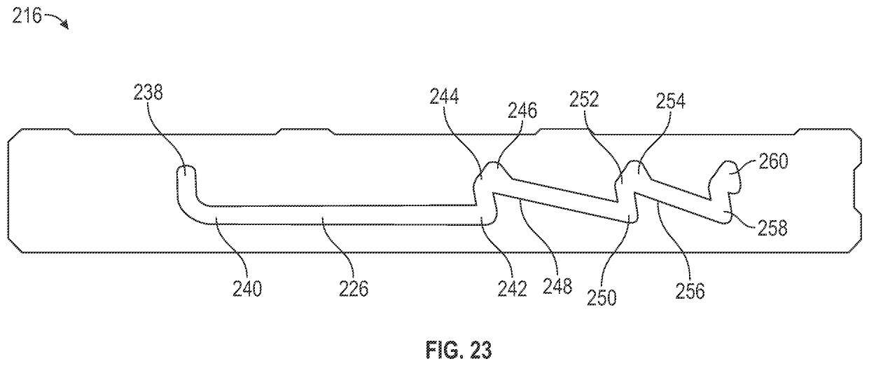

Referring to FIG. 23, an enlarged view of lock plate 216 is shown to explain the operation of device 200 (in combination with FIGS. 19-22.) Device 200 starts with pin 224 (FIG. 20) in distal-most location 238 in slot 226. Button 212 and pin 224 are biased upwardly by cantilever rods 228 into location 238. The vertical nature of slot 226 at location 238 prevents button 212 from being slid forward/distally. Once button 212 is depressed, pin 224 moves to location 240, thereby unlocking button 212 and permitting it to be moved forward. Button 212 may be moved forward until pin 224 reaches location 242, which stops button 212 from moving further forward. When button 212 is slid forward it drives cantilever shuttle 220 and mandrel 231 forward. At this location, the distal end of mandrel 231 contacts the proximal end of the implant (not shown.) Once button 212 is released, cantilever rod springs 228 urge button 212 and pin 224 upward to location 244. A ramped surface at this location drives button 212, shuttle 220 and mandrel 231 farther forward until pin 224 reaches location 246. At this location, device 200 is primed to dispense the first implant. When button 212 is again depressed, pin 224 contacts ramped surface 248 which causes button 212, shuttle 220 and mandrel 231 to be driven farther forward to location 250 in slot 226. At this point, the first implant has been dispensed. When button 212 is released, pin 224 travels to location 252 and then 254, in the same manner as it traveled to locations 244 and 246. At this location, device 200 is primed to dispense the second implant. When button 212 is again depressed, pin 224 contacts ramped surface 256 which causes button 212, shuttle 220 and mandrel 231 to be driven farther forward to location 258 in slot 226. At this point, the second implant has been dispensed. When button 212 is again released, it is urged upward into location 260. At this location, further pressing of button 212 results in no further longitudinal movement. At each stage along slot 226, the longitudinal movement of shuttle 220 may be displayed to the user by sticker 234 (see FIG. 22), as previously described.

While exemplary embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the invention and that methods and structures within the scope of these claims and their equivalents be covered thereby.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the disclosure. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and/or methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims. When a feature is described as optional, that does not necessarily mean that other features not described as optional are required.

The examples and illustrations included herein show, by way of illustration and not of limitation, specific embodiments in which the subject matter may be practiced. As mentioned, other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An implant delivery device comprising:
an elongated body having a proximal end and a distal end;
a cannula carried by the body and having a distal portion extending from the distal end of the body, the cannula being sized and configured to receive an implant therein;
a mandrel slidably received within the cannula and movable from a retracted position, in which an implant is retained inside the cannula, to a distally advanced position, in which the implant is ejected from the cannula by the mandrel;
a front shuttle coupled to the mandrel and configured to drive the mandrel from the retracted position to the distally advanced position;
a dispense button movable from an extended position, in which the mandrel remains in the retracted position, to a depressed position, in which the mandrel is moved toward the distally advanced position; and
a leaf spring, at least a portion of which spans between the dispense button and the front shuttle,
wherein the delivery device is configured to be operated by a user moving the dispense button from the extended position to the depressed position, thereby deflecting the leaf spring and urging the front shuttle towards the distal end of the body and causing the mandrel to move from the retracted position to the distally advanced position to eject the implant from the distal portion of the cannula.

2. The device of claim 1, wherein the dispense button is located on a top side surface of the elongated body and generally moves radially inward when moving from the extended position to the depressed position.

3. The device of claim 1, wherein the device further comprises a rear shuttle that is separately movable with respect to the front shuttle.

4. The device of claim 3, wherein the leaf spring spans between the front shuttle and the rear shuttle and has a mid-portion that contacts the dispense button.

5. The device of claim 3, wherein the leaf spring is pivotably connected to the front shuttle with a first pin and pivotably connected to the rear shuttle with a second pin.

6. The device of claim 3, wherein the dispense button comprises a cam surface that contacts a mating cam surface on the rear shuttle to drive the rear shuttle distally when the dispense button is moving from the extended position to the depressed position, thereby urging the leaf spring, the front shuttle and the mandrel further towards the distal end of the body.

7. The device of claim 3, wherein the rear shuttle comprises a locking feature that engages with a mating locking feature on the dispense button to lock the dispense button in the depressed position.

8. The device of claim 3, wherein the front shuttle and the rear shuttle have portions that overlap one another, and wherein the device further comprises a lock pin that extends from outside the elongated body and into the overlapping shuttle portions to prevent the front shuttle and the rear shuttle from moving relative to the body until the lock pin is removed.

9. The device of claim 8, wherein the device further comprises a cap configured to releasably cover the distal end of the body and the distal portion of the cannula when the cap is attached to the body, the cap being configured to be locked onto the body until the lock pin is removed.

10. The device of claim 8, wherein the device further comprises a sharps protection slide that is movable from a retracted position to a distally extended position in which the slide at least partially covers a distal tip of the cannula.

11. The device of claim 10, wherein the sharps protection slide covers a bottom portion of the cannula along a longitudinal length extending at least to the distal tip of the cannula and leaves a top portion visually exposed but not reachable by a flat object when the slide is in the extended position.

12. The device of claim 10, wherein the slide is configured to be locked in the retracted position until the lock pin is removed.

13. The device of claim 1, wherein the shuttle and the leaf spring are configured to return the dispense button to the extended position if the dispense button is not fully moved to the depressed position.

14. The device of claim 13, wherein the device is configured to emit a soft click when the dispense button reaches the depressed position.

15. The device of claim 1, wherein the cannula is provided with a retaining feature configured to releasably retain an implant within the cannula until the implant is pushed out by the mandrel.

16. The device of claim 15, wherein the retaining feature comprises a cutout section in the cannula which exposes at least a portion of the implant when loaded in the cannula, and a retention member configured to extend into the cutout section and urge the implant against an opposite wall of the cannula.

17. The device of claim 16, wherein the retention member is a flexible arm that puts a resilient force on the implant.

18. The device of claim 17, wherein the flexible arm is mounted on a retention hub that concentrically surrounds a portion of the cannula.

19. The device of claim 17, wherein the cutout section in the cannula has a longitudinal length that is nominally the same as a longitudinal length of the implant.

20. The device of claim 19, wherein the flexible arm comprises an implant contact surface having a longitudinal length that is nominally the same as the longitudinal lengths of the implant and the cutout section.

21. The device of claim 1, wherein the device further comprises a preloaded implant.

22. An implant delivery device comprising:
an elongated body having a proximal end, a distal end and a longitudinal axis;
a cannula carried by the body and having a distal portion extending from the distal end of the body, the cannula being sized and configured to receive an implant therein;
a mandrel slidably received within the cannula and movable from a retracted position, in which an implant is retained inside the cannula, to a distally advanced position, in which the implant is ejected from the cannula by the mandrel;
a front shuttle movable in a longitudinal direction and coupled to the mandrel, the front shuttle configured to drive the mandrel distally from the retracted position to the distally advanced position;
a rear shuttle movable in a longitudinal direction separately from the front shuttle;
a dispense button located on a top side surface of the elongated body and generally movable in a radially inward direction when moving from an extended position to a depressed position;
a leaf spring having a first end pivotably connected to the front shuttle, a second end pivotably connected to the rear shuttle, and a convex mid-portion contacting a surface of the dispense button such that when the button is moved toward the depressed position, the leaf spring becomes less curved and urges the front shuttle apart from the rear shuttle, wherein the dispense button comprises a cam surface that contacts a mating cam surface on the rear shuttle to drive the rear shuttle and leaf spring distally when the dispense button is moving towards the depressed position, wherein the rear shuttle comprises a locking feature that engages with a mating locking feature on the dispense button to lock the dispense button in the depressed position;
a cap configured to releasably cover the distal end of the body and the distal portion of the cannula when the cap is attached to the body;
a sharps protection slide that is movable from a retracted position to a distally extended position in which the slide at least partially covers a distal tip of the cannula; and
a lock pin configured to pass through the cap, the sharps protection slide, the rear shuttle and the front shuttle to prevent these components from moving until the lock pin is removed,
wherein the delivery device is configured to be operated by a user removing the lock pin, removing the cap, moving the dispense button from the extended position to the depressed position, thereby moving the rear shuttle distally and deflecting the leaf spring, both of which urge the front shuttle towards the distal end of the body and cause the mandrel to move from the retracted position to the distally advanced position to eject the implant from the distal portion of the cannula, and moving the sharps protection slide from the retracted position to the distally extended position.

23. The device of claim 22, wherein the first shuttle, the second shuttle and the leaf spring are configured to return the dispense button to the extended position if the dispense button is not fully moved to the depressed position.

24. The device of claim 23, wherein the device is configured to emit a soft click when the dispense button reaches the depressed position.

25. The device of claim 22, wherein the device further comprises a retention hub that concentrically surrounds a portion of the cannula, the retention hub having a flexible arm that extends through a cutout section in the cannula to resiliently urge an implant inside the cannula against an opposite wall of the cannula, thereby releasably retaining the implant within the cannula until the implant is pushed out by the mandrel.

26. A method of delivering an implant, the method comprising:
providing an implant delivery device including:
an elongated body with a proximal end and a distal end,
a cannula carried by the body and having a distal portion extending from the distal end of the body, the cannula carrying an implant therein,
a mandrel slidably received within the cannula,
a front shuttle coupled to the mandrel,
a dispense button located on the elongated body,
a leaf spring, at least a portion of which spans between the dispense button and the front shuttle,
a cap removably attached to the distal end of the body and covering the distal portion of the cannula when the cap is attached to the body, and
a lock pin configured to pass through the cap and the front shuttle to prevent these components from moving until the lock pin is removed;
removing the lock pin;
removing the cap;
inserting a distal tip of the cannula into a patient; and
moving the dispense button from an extended position to a depressed position, thereby deflecting the leaf spring which urges the front shuttle towards the distal end of the body and causes the mandrel to move from a retracted position to a distally advanced position to eject the implant from the distal portion of the cannula.

27. The method of claim 26, further comprising moving a sharps protection slide from a retracted position to a distally extended position in which the slide at least partially covers the distal tip of the cannula.

28. The method of claim 26, wherein the implant is ejected intravitreally.
